(19) United States
(12) Patent Application Publication
(10) Pub. No.: US 2024/0325623 A1
(43) Pub. Date: Oct. 3, 2024

(54) DOUBLE LAYER TUBING FOR IV INFUSION SETS

(71) Applicant: CareFusion 303, Inc., San Diego, CA (US)

(72) Inventors: Fanqing Meng, Buffalo Grove, IL (US); Mark Doane, North Fort Myers, FL (US); Marc William Weimer, South Jordan, UT (US)

(21) Appl. No.: 18/593,175

(22) Filed: Mar. 1, 2024

Related U.S. Application Data
(60) Provisional application No. 63/455,207, filed on Mar. 28, 2023.

(51) Int. Cl. A61M 5/14

(52) U.S. Cl. CPC A61M 5/14

(57) ABSTRACT

A continuous tube includes an inner layer comprising a polyethylene composition and an outer layer comprising a thermal plastic elastomer. The inner layer and the outer layer are concentrically disposed without having an intermediate layer therebetween.

### CROSS REFERENCE TO RELATED APPLICATION

This application claims the benefit of priority under 35 U.S.C. § 119 to U.S. Provisional Patent Application Ser. No. 63/455,207, entitled “DOUBLE LAYER TUBING FOR IV INFUSION SETS”, filed on Mar. 28, 2023, the entire contents of which is incorporated herein by reference.

### BACKGROUND

### Field

The present disclosure relates to the field of medication delivery. More specifically, the present disclosure relates to multi-layer infusion tubing used for delivering medication.

### Background

Tubing lines used for delivering medication to patients (e.g., via intravenous infusion) are often required to meet stringent limits relating to drug adsorption and performance requirements such as kinking resistance, transparency, surface feeling, and the like.

Conventional infusion tubing is formed of polyethylene-lined polyvinyl chloride (PE-lined PVC), and typically requires a tie layer such as ethyl vinyl acetate to bond the non-polar PE to polar PVC to prevent delamination. There are several disadvantages of using EVA-based tie layer adhesives in such a system. One main drawback is that EVA gives off an odor when extruded at high temperatures. EVA resins are known for their low temperature stability. Additionally, EVA resins degrade at high processing temperatures, contaminate the extrusion system, and create gels and black specks in the laminates.

### SUMMARY

The techniques disclosed herein advantageously can provide improved, PVC-free infusion tubing and eliminate the regulatory and environmental risk of using PVC and the plasticizers in infusion tubing for delivery of medications. Further advantageously, the tubing disclosed herein offers low drug absorption so as to meet labelling statement of PE-line infusion tubing from some drug's instructions for use. Furthermore, the coated PE material improves tubing spallation resistance as a pumping tubing segment by reducing TPE inner surface friction.

While TPE has no small molecular plasticizer as being presented in PVC, TPE may still include oils, slipping agent and other small quantity of additives, which can be potentially leach into the fluids being infused through the tubing. Advantageously, the elimination of PVC and the coating of PE results in prevention of chemical leaching from TPE into administrated drug solutions.

The methods disclosed herein advantageously and significantly simplify the extrusion processing by eliminating the tie-layer due to compatibility between PE and TPE. In addition, disclosed methods provide inexpensive techniques for providing infusion tubing that eliminates potential loss of medication through adsorption or absorption on the inner surface of the tubing while maintaining performance metrics such as kinking resistance and surface feel. Even though specific use examples include the medical industry, these solutions have wide application potentials in other industries.

In one embodiment, a continuous tube includes an inner layer comprising a polyethylene, and an outer layer comprising a thermal plastic elastomer. The inner layer and the outer layer are concentrically disposed without having an intermediate layer therebetween.

In another embodiment, an infusion set includes a continuous tube disclosed herein bound to a medical connector.

In another embodiment, a method of preparing a continuous tube includes co-extruding a polyethylene composition with a thermal plastic elastomer resin without providing an adhesive or a tie layer therebetween.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 shows a schematic cross-section of a tube in accordance with at least some embodiments of the present disclosure.

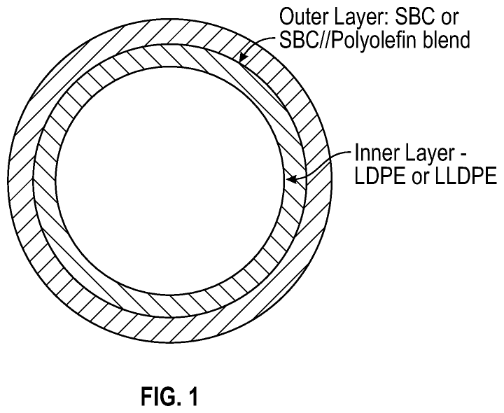

FIGS. 2A-2B show cross-section of examples of a tube in accordance with at least some embodiments of the present disclosure.

FIGS. 3A-3B show images of two types of conventional PE-lined blue tinted PVC tube with a tie layer following a certain number of activation-deactivation cycles using a pinch clamp.

FIGS. 4A-4B show images of two different types of PE-lined TPE tubes following the same number of activation-deactivation cycles using a pinch clamp as in FIGS. 3A-3B.

FIGS. 5A-5B show images of two types of PE-lined blue tinted PVC tube with a tie layer following a certain number of activation-deactivation cycles using a roller clamp.

FIGS. 6A-6B show images of two different types of PE-lined TPE tubes following the same number of activation-deactivation cycles using a roller clamp as in FIGS. 5A-5B.

FIG. 7 shows an image comparing the results of a blade-scratching test on the two types of PE-lined blue tinted PVC tubes shown in FIGS. 3A-3B and the two different types of PE-lined TPE tubes shown in FIGS. 4A-4B.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for case of understanding.

Conventionally, the tubing used for intravenous delivery of medications is made from polyvinyl chloride (PVC) and is lined on the inside with non-polar polymer materials so prevent interaction between PVC and the drug molecules. The mostly commonly used non-polar polymer for lining a PVC tubing is polyethylene (PE), which does not typically have good adhesion characteristics with PVC. Consequently, a tie layer is used for bonding the non-polar PE material to the PVC tube to prevent delamination.

An example of a material used for the tie layer is ethyl vinyl acetate (EVA). However, EVA has several drawbacks when used in a medical application. For example, EVA resins give off an odor when extruded at high temperatures. While EVA resins are known for their low temperature stability, they degrade at high processing temperatures and thus, contaminate the extrusion system by creating gels and black specks in the laminates. Consequently, an alternative tubing structure is desired for simplifying the extrusion process and eliminating the concerns relating to tie layer chemistry by providing materials that do not require a tie layer for bonding.

Accordingly, in an aspect of the present disclosure, a continuous double layer tube without a tie layer is provided. The continuous tube includes an inner layer including a polyethylene (PE) composition, and an outer layer including a thermal plastic elastomer (TPE). The inner layer and the outer layer are concentrically disposed without having in intermediate layer therebetween.

The materials of the inner layer and the outer layer are selected to be free of polyvinyl chloride. Consequently, the continuous tubing disclosed herein is free of polyvinyl chloride, and thus, may be used for delivery of drug molecules that have stringent instructions for use (IFU) requirements.

In some embodiments, the inner layer includes a low-density polyethylene (LDPE). LDPE can be made in translucent and opaque variations depending on particular applications. For example, in some embodiments, the tubing may be required for delivery of light-sensitive drug molecules. In such applications, an opaque variation of the LDPE may be used as the inner layer. In some embodiments, the inner layer is transparent to visible light.

In some embodiments, the inner layer includes a linear low-density polyethylene (LLDPE) such as, for example, a copolymer of ethylene and alpha-olefin. Non-limiting examples of alpha-olefins that may be used in LLDPE include butene, hexene, and octene. In some embodiments, the inner layer includes a copolymer of ethylene and octene.

In some embodiments, the TPE of the outer layer includes a styrenic block copolymer. Non-limiting examples of a styrenic block copolymer include styrene-butadiene-styrene (SBS), styrene-ethylene-butylene-styrene (SEBS), styrene-isoprene block copolymer (SIS), styrene-ethylene-propylene-styrene (SEPS), styrene-ethylene-ethylene-propylene-styrene (SEEPS), or a combination thereof.

In some embodiments, the TPE of the outer layer may be blended with a polyolefin such as, for example, a polypropylene or a polyethylene composition depending on the particular applications for which the tubing is to be used.

In some embodiments, the outer layer may further include an additive. Some examples of additives include, but are not limited to, a slipping agent, an antioxidant, an ultraviolet light and blue light blocking additive, or a combination thereof. In some embodiments, the additive may include a light blocking additive that renders the tubing opaque to visible and/or ultraviolet light. In some embodiments, the outer layer is transparent to visible light.

While the conventional multilayer tubing has a relatively thin inner layer, the continuous tubing of the present disclosure may be made with any suitable ratio of thicknesses of the inner layer and the outer layer. In some embodiments, the inner layer may have a thickness in a range from about 0.1% to about 20% of the thickness of the outer layer. For example, the ratio of the thickness of the inner layer to the thickness of the outer layer may be in a range of about 0.1%, about 0.2%, about 0.3%, about 0.4%, about 0.5%, about 0.6%, about 0.7%, about 0.8%, about 0.9%, about 1.0%, about 1.25%, about 1.5%, about, 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, or any other ratio between any two of these ratios.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to 5 percent of the actual value stated, and other suitable tolerances.

In some embodiments, the thickness of the inner layer may be in a range from about 0.01 mm to about 1 mm. Thus, for example, the thickness of the inner layer may be about 0.01 mm, about 0.02 mm, about 0.03 mm, about 0.04 mm, about 0.05 mm, about 0.06 mm, about 0.07 mm, about 0.08 mm, about 0.09 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, about 1.0 mm, or any thickness between any two of these thicknesses.

In some embodiments, the thickness of the outer layer may be in a range from about 0.05 mm to about 10 mm. Thus, for example, the thickness of the outer layer may be about 0.05 mm, about 0.06 mm, about 0.07 mm, about 0.08 mm, about 0.09 mm, about 0.1 mm, about 0.125 mm, about 0.15 mm, about 0.175 mm, about 0.2 mm, about 0.225 mm, about 0.25 mm, about 0.275 mm, about 0.3 mm, about 0.325 mm, about 0.35 mm, about 0.375 mm, about 0.4 mm, about 0.425 mm, about 0.45 mm, about 0.475 mm, about 0.5 mm, about 0.525 mm, about 0.55 mm, about 0.575 mm, about 0.6 mm, about 0.625 mm, about 0.65 mm, about 0.675 mm, about 0.7 mm, about 0.725 mm, about 0.75 mm, about 0.775 mm, about 0.8 mm, about 0.825 mm, about 0.85 mm, about 0.875 mm, about 0.9 mm, about 0.925 mm, about 0.95 mm, about 0.975 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10.0 mm, or any thickness between any two of these thicknesses.

The outer layer is disposed concentrically over the inner layer in the continuous tubing such that the outer layer is in direct contact with the inner layer without having an intermediate layer therebetween. In other words, there is no adhesive or tie layer between the inner layer and the outer layer.

Without wishing to be bound by a particular theory, delamination of the inner layer from the outer layer may be prevented by selecting the corresponding materials to have similar tensile properties. In some embodiments, the tensile elongations of the materials of the inner and outer layers may be within about 10% of each other. For example, the tensile elongation at break for the inner layer may be in a range from about 500% to about 700%, or about 600%, or about 650%. Likewise, the tensile elongation at break for the outer layer may be in a range from about 500% to about 700%, or in a range from about 600% to about 700%, or in a range from about 620% to about 670%.

In some embodiments, the material of the inner layer is selected to have low adsorption of nitroglycerin and/or other drug molecules. For example, the material of the inner layer is selected such that the amount of nitroglycerin adsorbed to the material is about 10% or less than the amount of nitroglycerin adsorbed to polyvinyl chloride. In some embodiments, the material of the inner layer is specifically selected to have low adsorption and reactivity to one or more drugs selected from Aldesleukin, Calcitriol, Carmustine, Chlordiazepoxide HCl, Diazepam, Insulin, Isosorbide Dinitrate, Lorazepam, Miconazole, Nitroglycerin, Sufentanil Citrate, Thiopental Sodium, Urokinase, and Warfarin Sodium.

Thus, according to another aspect of the present disclosure, an infusion set for infusion of medicaments is provided. The infusion set may include a continuous tubing disclosed herein, and a medical connector. Medical connectors that can benefit from tubing of the present disclosure include, without limitation, connectors made of acrylic-based polymeric materials such as acrylonitrile-butadiene-styrene (ABS), methyl methacrylate-acrylonitrile-butadiene-styrene (mABS), acrylic-polycarbonate based materials, polyester, polycarbonate, acrylic, etc.

In some embodiments, the infusion set may include the continuous tubing according to the present disclosure that is mated and bond to a medical connector by solvent boding. In some embodiments, solvent bonding includes applying a thin layer of solvent on one or both of the contacting surfaces, e.g., a surface of the tubing and a surface of the medical connector, and then bringing the two surfaces together and holding in place until a seal is formed. The contacting surface of the tubing can either be at the inner diameter, the outer diameter, or both diameters depending on whether the adhesion layer is an inner layer, outer layer or both. The solvent is applied either internally, externally, or both.

Solvents that can be used in solvent bonding tubing of the present disclosure include, without limitation, one or more of an alcohol, such as methanol, ethanol, isopropanol, propanol, butanol, pentanol, benzyl alcohol, a glycol such as diethylene glycol, propylene glycol, an ether, such as dioxolane, tetrahydrofuran, 1,3-dioxane, a ketone, such as acetone, methyl ethyl ketone, cyclohexanone, an ester such as ethyl acetate, ethyl formate, an amide such as dimethyl formamide, dimethyl and diethyl acetamide, etc.

According to another aspect of the present disclosure, a method of manufacturing a continuous tubing disclosed herein is provided. In some embodiments, the method includes co-extruding a polyethylene composition with a thermal plastic elastomer (TPE) resin without providing an adhesive or a tie layer therebetween. Put another way, the polyethylene composition and the TPE resin are co-extruded to form a continuous tubing such that a layer polyethylene composition is concentrically formed on an inner wall of a circular layer formed by the TPE resin, without having any adhesive or tie layer disposed therebetween.

The materials of the inner layer and the outer layer are selected to be free of polyvinyl chloride. Thus, in some embodiments, the inner layer may be a low-density polyethylene (LDPE) or a linear low density polyethylene (LLDPE).

In some embodiments, the TPE resin of the outer layer includes a styrenic block copolymer. Non-limiting examples of a styrenic block copolymer include styrene-butadiene-styrene (SBS), styrene-ethylene-butylene-styrene (SEBS), styrene-isoprene block copolymer (SIS), styrene-ethylene-propylene-styrene (SEPS), styrene-ethylene-ethylene-propylene-styrene (SEEPS), or a combination thereof. In some embodiments, the TPE of the outer layer may be blended with a polyolefin such as, for example, a polypropylene or a polyethylene composition depending on the particular applications for which the tubing is to be used.

In some embodiments, the outer layer may further include an additive such as, for example, a slipping agent, an antioxidant, an ultraviolet light blocking additive and blue light blocking additive or a combination thereof. In some embodiments, the additive may include a light blocking additive that renders the tubing opaque to visible and/or ultraviolet light. In some embodiments, the material of the outer layer is selected such that the outer layer is transparent to visible light.

The inner layer may have a thickness in a range from about 0.1% to about 20% of the thickness of the outer layer. Thus, for example, the amount of polyethylene composition co-extruded with the TPE resin may enable formation of an inner layer having a thickness in a range from about 0.01 mm to about 1 mm. Likewise, the amount of TPE resin used during the manufacturing process may enable formation of an outer layer having a thickness in a range from about 0.05 mm to about 10 mm.

### EXAMPLES

A medical grade of Polyethylene was co-extruded with a TPE resin to form a continuous tubing. The selected PE grade was ENGAGE™ 8480K Health+ polyolefin elastomer. This grade is an ethylene-octene copolymer that offers high tensile elongation at 660%. Two TPE compositions (MD50278 and MD42275) were selected in this study from Teknor Apex with tensile elongation at 620% and 670%, respectively.

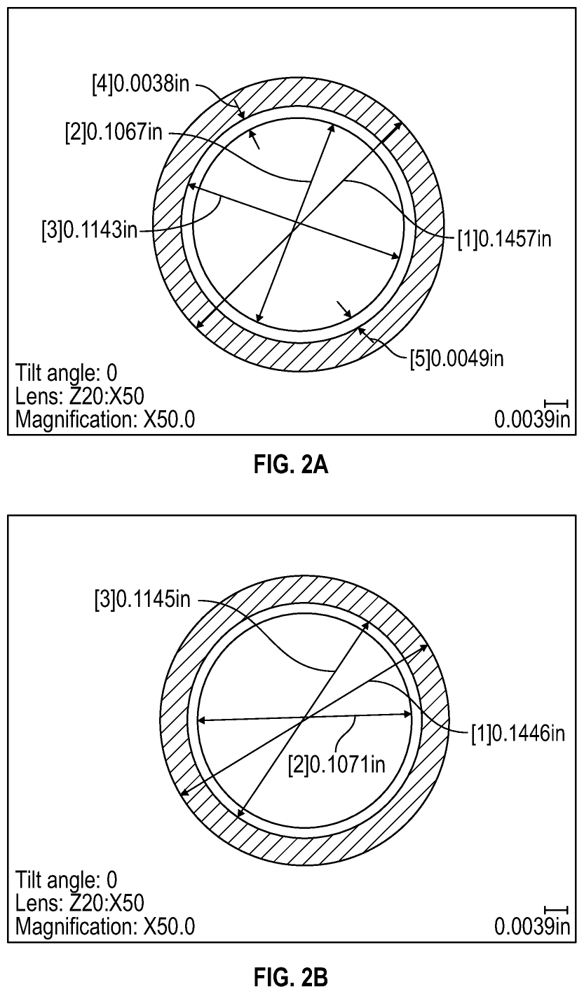

FIGS. 2A and 2B respectively show images of the cross-section of a PE-lined tubing with an MD50278 TPE (FIG. 2A) as the outer layer, and an MD42275 TPE (FIG. 2B) as the outer layer. The figures show that the PE inner layer has a thickness of about 0.0127 mm in FIG. 2A and 0.0106 mm in FIG. 2B.

To evaluate the durability of PE laminated on the inner TPE tubing substrate, two PE-lined blue tinted PVC tubing with a tie layer, labelled as TC50015844 and 10013072, were used for this study as a benchmark. The comparison was performed using two testing approaches:
(a) Activation and deactivation by pinch clamping or roller clamping 63 times to observe the delamination.
(b) Slit the tubing and scratch the inner surface with blade to attempt to peel off the PE layer from the tubing substrate for the durability observation.

The number 63 was selected to simulate the general use condition in hospital settings of 9 times a day for up to 7 days of providing the medication to a patient via infusion using the tubing.

### Example 1: Clamping Study

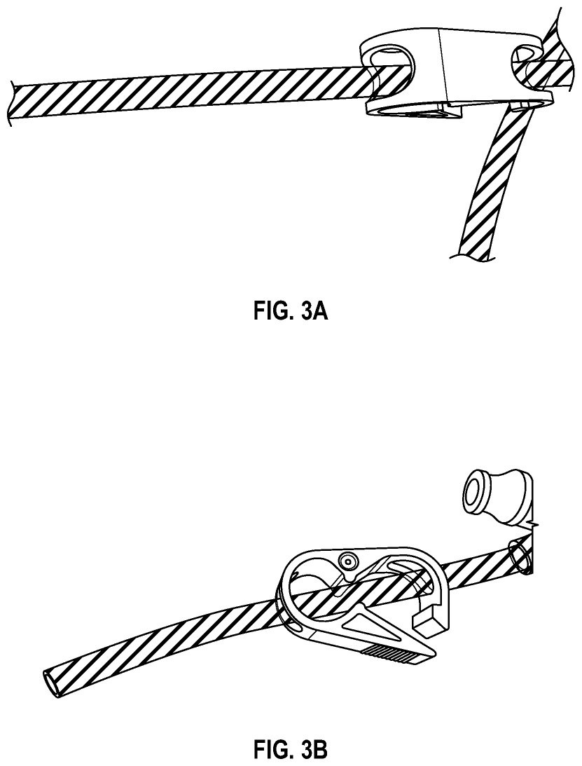

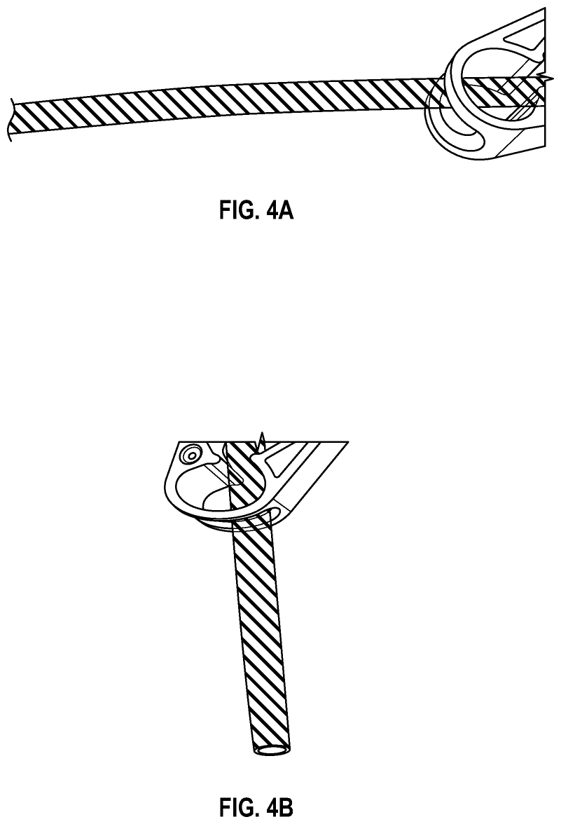

Flow in different tubes was activated or deactivated 63 times for each tube using a common pinch clamp. As can be seen in FIG. 3A (TC50015844 PE-lined PVC tube), FIG. 3B (10013072 PE-lined PVC tube), and FIG. 4A (PE-lined TPE MD50278), FIG. 4B (PE-lined TPE MD40275) this study showed excellent bondability between PE and TPE, compared to the PE-lined PVC tubes even without a tie-layer. The PE-lined TPE tubes did not show any sign of delamination.

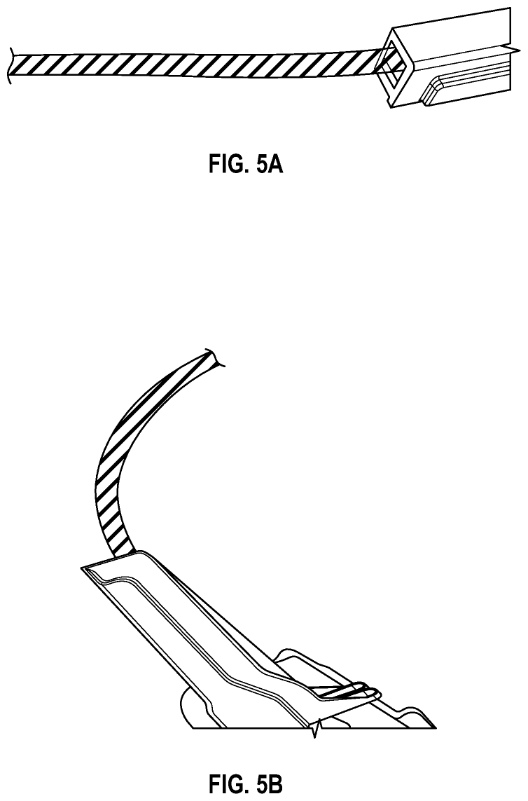

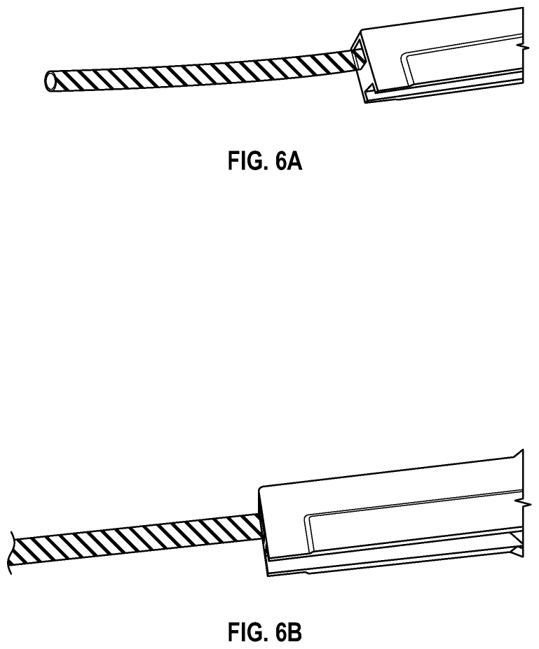

In a separate study, flow in the tubes was activated or deactivated 63 times for each tube using a common roller clamp. FIGS. 5A-5B (TC50015844 PE-lined PVC tube and 10013072 PE-lined PVC tube respectively) and FIGS. 6A-6B (PE-lined TPE MD50278 and PE-lined TPE MD40275 respectively) show that the delamination testing after 63 times activation and deactivation cycles using a roller clamp did not show any sign of delamination.

### Example 2: Peeling Study

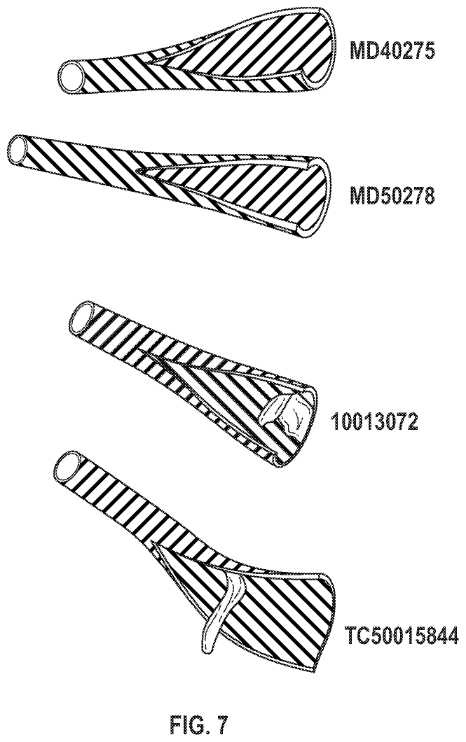

Different tubes were slit open to expose the inner surface, and inner surface was then scratched with a blade. FIG. 7 shows a comparison between PE-lined TPE tubes (labeled MD50278 and MD40275) and PE-lined PVC tubes (labeled TC50015844 and 10013072). As can be seen in FIG. 7 testing the inner layer by aggressively scratching with a blade demonstrated a peeled layer from two PE-lined PVC samples, but no delamination was observed from PE-lined TPE. These testing results demonstrate that PE has strong bonding to TPE substrate even without a tie layer.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1: A continuous tube comprising: an inner layer comprising a polyethylene composition; and an outer layer comprising a thermal plastic elastomer (TPE), wherein the inner layer and the outer layer are concentrically disposed without having an intermediate layer therebetween.
Clause 2: The continuous tube of clause 1, wherein the inner layer comprises a low-density polyethylene or a copolymer of ethylene and alpha-olefin.
Clause 3: The continuous tube of clause 2, wherein the inner layer comprises an ethylene-octene copolymer.
Clause 4: The continuous tube of any one of the preceding clauses, wherein the TPE comprises a styrenic block copolymer.
Clause 5: The continuous tube of clause 4, wherein the TPE comprises styrene-butadiene-styrene (SBS), styrene-ethylene-butylene-styrene (SEBS), styrene-isoprene block copolymer (SIS), styrene-ethylene-propylene-styrene (SEPS), styrene-ethylene-ethylene-propylene-styrene (SEEPS), or a combination thereof.
Clause 6: The continuous tube of clause 4, wherein the TPE is blended with a polyolefin.
Clause 7: The continuous tube of any one of the preceding clauses, wherein the outer layer further comprises one or more of a slipping agent, an antioxidant, and an ultraviolet light blocking additive.
Clause 8: The continuous tube of any one of the preceding clauses, wherein the inner layer has a thickness in a range from 0.01 mm to 1 mm.
Clause 9: The continuous tube of any one of the preceding clauses, wherein the polyethylene material of the inner layer and the TPE material of the outer layer have tensile elongation within 10% of each other.
Clause 10: The continuous tube of any one of the preceding clauses, wherein inner layer and the outer layer are resistant to delamination following pinch clamping or roller clamping.
Clause 11: The continuous tube of any one of the preceding clauses, wherein inner layer exhibits nitroglycerin adsorption that is less than 10% of nitroglycerin adsorption exhibited by polyvinyl chloride.
Clause 12: The continuous tube of any one of the preceding clauses, wherein the continuous tube is medical tubing for administration of a medical fluid by infusion.
Clause 13: The continuous tube of any one of the preceding clauses, wherein the continuous tubing is polyvinyl chloride free.
Clause 14: The continuous tube of any one of the preceding clauses, wherein the inner layer has thickness that is from about 1% to about 20% of a thickness of the outer layer.
Clause 15: The continuous tube of any one of the preceding clauses, wherein the continuous tubing is transparent to visible light.
Clause 16: An infusion set comprising the continuous tube of clause 1, bound to a medical connector.
Clause 17: The infusion set of clause 16, wherein the infusion set is used for infusing a drug selected from the group consisting of: Aldesleukin, Calcitriol, Carmustine, Chlordiazepoxide HCl, Diazepam, Insulin, Isosorbide Dinitrate, Lorazepam, Miconazole, Nitroglycerin, Sufentanil Citrate, Thiopental Sodium, Urokinase, and Warfarin Sodium.
Clause 18: A method of preparing the continuous tube of clause 1, the method comprising: co-extruding a polyethylene composition with a thermal plastic elastomer resin without providing an adhesive or a tie layer therebetween.
Clause 19: The method of clause 18, wherein the polyethylene composition comprises a low-density polyethylene or a copolymer of ethylene and alpha-olefin.
Clause 20: The method of clause 18, wherein the TPE resin comprises a styrenic block copolymer.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

In one or more aspects, the terms “about,” “substantially,” and “approximately” may provide an industry-accepted tolerance for their corresponding terms and/or relativity between items, such as from less than one percent to five percent.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A continuous tube comprising:
an inner layer comprising a polyethylene composition; and
an outer layer comprising a thermal plastic elastomer (TPE),
wherein the inner layer and the outer layer are concentrically disposed without having an intermediate layer therebetween.

2. The continuous tube of claim 1, wherein the inner layer comprises a low-density polyethylene or a copolymer of ethylene and alpha-olefin.

3. The continuous tube of claim 2, wherein the inner layer comprises an ethylene-octene copolymer.

4. The continuous tube of claim 1, wherein the TPE comprises a styrenic block copolymer.

5. The continuous tube of claim 4, wherein the TPE comprises styrene-butadiene-styrene (SBS), styrene-ethylene-butylene-styrene (SEBS), styrene-isoprene block copolymer (SIS), styrene-ethylene-propylene-styrene (SEPS), styrene-ethylene-ethylene-propylene-styrene (SEEPS), or a combination thereof.

6. The continuous tube of claim 4, wherein the TPE is blended with a polyolefin.

7. The continuous tube of claim 1, wherein the outer layer further comprises one or more of a slipping agent, an antioxidant, and an ultraviolet light blocking additive.

8. The continuous tube of claim 1, wherein the inner layer has a thickness in a range from 0.01 mm to 1 mm.

9. The continuous tube of claim 1, wherein the polyethylene material of the inner layer and the TPE material of the outer layer have tensile elongation within 10% of each other.

10. The continuous tube of claim 1, wherein inner layer and the outer layer are resistant to delamination following pinch clamping or roller clamping.

11. The continuous tube of claim 1, wherein inner layer exhibits nitroglycerin adsorption that is less than 10% of nitroglycerin adsorption exhibited by polyvinyl chloride.

12. The continuous tube of claim 1, wherein the continuous tube is medical tubing for administration of a medical fluid by infusion.

13. The continuous tube of claim 1, wherein the continuous tubing is polyvinyl chloride free.

14. The continuous tube of claim 1, wherein the inner layer has thickness that is from about 1% to about 20% of a thickness of the outer layer.

15. The continuous tube of claim 1, wherein the continuous tubing is transparent to visible light.

16. An infusion set comprising the continuous tube of claim 1, bound to a medical connector.

17. The infusion set of claim 16, wherein the infusion set is used for infusing a drug selected from the group consisting of: Aldesleukin, Calcitriol, Carmustine, Chlordiazepoxide HCl, Diazepam, Insulin, Isosorbide Dinitrate, Lorazepam, Miconazole, Nitroglycerin, Sufentanil Citrate, Thiopental Sodium, Urokinase, and Warfarin Sodium.

18. A method of preparing the continuous tube of claim 1, the method comprising:
co-extruding a polyethylene composition with a thermal plastic elastomer resin without providing an adhesive or a tie layer therebetween.

19. The method of claim 18, wherein the polyethylene composition comprises a low-density polyethylene or a copolymer of ethylene and alpha-olefin.

20. The method of claim 18, wherein the TPE resin comprises a styrenic block copolymer.
